(19) United States
(12) Patent Application Publication
(10) Pub. No.: US 2017/0183622 A1
(43) Pub. Date: Jun. 29, 2017

(54) BIOPAPERS AS A SUBSTRATE FOR TISSUE CULTURE

(71) Applicant: The Government of the US, as represented by the Secretary of the Navy, Arlington, VA (US)

(72) Inventors: Russell Kirk Pirlo, Fort Washington, MD (US); Peter K. Wu, Ashland, OR (US); Bradley R. Ringeisen, Lorton, VA (US); Lauren Bischel, Pewaukee, WI (US)

(73) Assignee: The Government of the United States of America, as represented by the Secretary of the Navy, Arlington, VA (US)

(21) Appl. No.: 15/368,049

(22) Filed: Dec. 2, 2016

Related U.S. Application Data
(60) Provisional application No. 62/262,644, filed on Dec. 3, 2015.

(51) Int. Cl. C12N 5/00

(52) U.S. Cl. CPC C12N 5/0068; C12N 2533/54; C12N 2537/10

(57) ABSTRACT

A biocompatible membranes made by electrospinning a polymer, having living cells on both surfaces of the membrane, and/or is free of a non-biodegradable structural component. The membranes may be attached to a frame and span a hole in the frame. A method of: providing a biocompatible membrane made by electrospinning a polymer and depositing living cells both surfaces of the membrane and/or attaching the membrane to a frame and spanning a hole in the frame. Different types of cells are deposited on each surface of the membrane.

This application claims the benefit of U.S. Provisional Application No. 62/262,644, filed on Dec. 3, 2015. The provisional application and all other publications and patent documents referred to throughout this nonprovisional application are incorporated herein by reference.

### TECHNICAL FIELD

The present disclosure is generally related to biopapers used as tissue culture substrates.

### DESCRIPTION OF RELATED ART

Despite the critical role the extracellular matrix (ECM) and cell-cell contact play in barrier tissues and the prevalence of the transwell platform for in vitro modeling of these tissues, addressing the deficiencies of the membrane component of the transwell system has received very little attention. Morris et al. have sought to mimic the 3D topography of the natural bronchiole environment by electrospinning nonwoven microfiber PET scaffolds and using them in a transwell co-culture of epithelial and fibroblast cells. These scaffolds employed a non-biological polymer. Nonetheless, electrospinning is a technique that can be used with a vast number of different polymers, and has been used for biomaterials. For example, electrospun micro- and nano-fiber mats are an attractive cell culture substrate as the fiber diameter (Sisson et al., “Fiber diameters control osteoblastic cell migration and differentiation in electrospun gelatin” J. Biomed. Mater. Res. A, vol. 94, no. 4, pp. 1312-1320, September 2010; Zhao et al., “Preparation and cytocompatibility of PLGA scaffolds with controllable fiber morphology and diameter using electrospinning method” J. Biomed. Mater. Res. B Appl. Biomater. vol. 87B, no. 1, pp. 26-34, 2008), alignment (Chang et al., “Cell orientation and regulation of cell-cell communication in human mesenchymal stem cells on different patterns of electrospun fibers” Biomed. Mater., vol. 8, no. 5, p. 055002, October 2013; Ren et al., “Enhanced differentiation of human neural crest stem cells towards the Schwann cell lineage by aligned electrospun fiber matrix” Acta Biomater., vol. 9, no. 8, pp. 7727-7736, August 2013; Ghasemi-Mobarakeh et al., “Electrospun poly(ε-caprolactone)/gelatin nanofibrous scaffolds for nerve tissue engineering” Biomaterials, vol. 29, no. 34, pp. 4532-4539, December 2008), and pore size (Lowery et al., “Effect of fiber diameter, pore size and seeding method on growth of human dermal fibroblasts in electrospun poly(ε-caprolactone) fibrous mats” Biomaterials, vol. 31, no. 3, pp. 491-504, January 2010) can each be tuned to optimize favorable migration, proliferation (Zhao), and differentiation responses by cells, including astrocytes (Qu et al., “Electrospun silk fibroin nanofibers in different diameters support neurite outgrowth and promote astrocyte migration” J. Biomed. Mater. Res. A, vol. 101A, no. 9, pp. 2667-2678, September 2013). Furthermore, degradable and biologically based polymers, such as gelatin, can be employed as the fiber material, enhancing adhesion, affording degradation with minimal byproducts, and offering ligand activation and remolding that may be beneficial to the blood-brain barrier (BBB) model (Ghasemi-Mobarakeh; Hajiali et al., “Electrospun PGA/gelatin nanofibrous scaffolds and their potential application in vascular tissue engineering” Int. J. Nanomedicine, p. 2133, September 2011; Ozeki et al., “In vivo degradability of hydrogels prepared from different gelatins by various cross-linking methods” J. Biomater. Sci. Polym. Ed., vol. 16, no. 5, pp. 549-561, January 2005; Baiguera et al., “In vitro astrocyte and cerebral endothelial cell response to electrospun poly(ε-caprolactone) mats of different architecture” J. Mater. Sci. Mater. Med., vol. 21, no. 4, pp. 1353-1362, December 2009).

Several groups have used electrospinning to fabricate gelatin-based cell culture substrates, investigating not only the influence of mechanical characteristics on cell migration and differentiation but also on the biocompatibility/viability effects of different cross-linking agents (Sisson et al., “Evaluation of cross-linking methods for electrospun gelatin on cell growth and viability” Biomacromolecules, vol. 10, no. 7, pp. 1675-1680, July 2009; Panzavolta et al., “Electrospun gelatin nanofibers: Optimization of genipin cross-linking to preserve fiber morphology after exposure to water” Acta Biomater., vol. 7, no. 4, pp. 1702-1709; Jalaja et al., “Modified dextran cross-linked electrospun gelatin nanofibres for biomedical applications” Carbohydr. Polym., vol. 114, pp. 467-475, December 2014, April 2011).

### BRIEF SUMMARY

Disclosed herein is an article comprising: a biocompatible membrane made by electrospinning a polymer and a rigid component having a hole. The membrane is attached to the rigid component and spans the hole.

Also disclosed herein is an article comprising: a biocompatible membrane made by electrospinning a polymer and living cells on both surfaces of the membrane. The surfaces of the membrane comprise different types of cells.

Also disclosed herein is an article comprising: a biocompatible membrane comprising a polymer that is free of a non-biodegradable structural component and a rigid component having a hole. The membrane is attached to the rigid component and spans the hole.

Also disclosed herein is a method comprising: providing a biocompatible membrane made by electrospinning a polymer and attaching the membrane to a rigid component having a hole. The membrane spans the hole.

Also disclosed herein is a method comprising: providing a biocompatible membrane made by electrospinning a polymer and depositing living cells both surfaces of the membrane. Different types of cells are deposited on each surface of the membrane.

### BRIEF DESCRIPTION OF THE DRAWINGS

A more complete appreciation will be readily obtained by reference to the following Description of the Example Embodiments and the accompanying drawings.

FIGS. 1A-B show cross-sections of a triple layer biopaper before (FIG. 1A) and after (FIG. 1B) crosslinking.

FIG. 2 shows a gelatin biopaper attached to a COC frame with expanded PTFE gasket.

FIG. 3 shows a biopaper attached to a stainless steel frame, shown with a bioreactor into which it is placed.

FIG. 4 shows an image of an inverted standard PET cell culture insert with the center of the membrane removed.

FIG. 5 shows an image of 15% electrospun and cross-linked gelatin biopaper attached to the bottom of the cell culture insert.

FIG. 6 shows a schematic side-view of the in vitro bilayer human blood-brain barrier model.

FIGS. 7A-D show 5000× micrographs of fibers using 15% vs 30% gelatin as spun. FIG. 7A is of a fresh 30% solution. FIG. 7B is of a 30% solution that was electrospun 24 hours after preparation. FIG. 7C is of a fresh 15% solution. FIG. 7D is of a 15% solution that was electrospun 24 hours after preparation. The scale bar is 5 μm.

FIGS. 8A-B show 3000× micrographs of fibers using glyceraldehyde (FIG. 8A) vs genipin soak (30%) (FIG. 8B). The scale bar is 5 μm.

FIG. 9 shows 5000× a micrograph of fibers made with no genipin in gelatin solution (30%, cross-linked after). The scale bar is 5 μm.

FIGS. 10A-B show 5000× micrographs of fibers soaked in genipin for 4 days (FIG. 10A) and 7 days (FIG. 10B) and exposed to water. The scale bar is 5 μm.

FIGS. 11A-F show confocal microscopy images of a BBB model for electrospun biopaper (FIGS. 11A-C) and standard PET (FIGS. 11D-F). FIGS. 11A and 11D show the endothelial layer (stained for CD31) and FIGS. 11B and 11E show the astrocyte layer (stained for GFAP). Volume-rendered side views (FIGS. 11C and 11F) show that there is virtually no gap between the layers in the biopaper supported model (FIG. 11C) whereas there is a definite gap between layers supported by a standard polymer membrane (FIG. 11F).

FIG. 12 shows TEER values. The model supported by 15% gelatin formulation of biopaper attained more stable TEER readings which were higher than those on PET by day 14.

FIGS. 13A-C show confocal microscopy images of a bilayer lung tissue model supported by 15% gelatin biopaper. FIG. 13A: HMVEC-L stained for CD31. FIG. 13B: SAEC stained for MUCINSAC. FIG. 13C: volume-rendered side view.

### DETAILED DESCRIPTION OF EXAMPLE EMBODIMENTS

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

Disclosed herein is a cell culture substrate based on electrospinning fibers into thin, porous mats. Specifically a method has been developed to create electrospun gelatin biopapers as a substrate for in vitro bilayer models of barrier tissues such as the blood brain barrier, the lung, the gut, and any other barrier tissue culture or tissue models. In these models the electrospun biopapers simulate the basal membrane present between two or more cell types that comprise the barrier tissue. These electrospun biopapers may also be used as previously described in U.S. Pat. No. 8,669,086, where the biopapers support formation of 3D tissues by stacking multiple tissue constructs built upon the biopaper. A potential improvement to this biopaper is the use of electrospinning to create the underlying membrane. The previous method involved porogen leaching, casting/molding, or laser milling.

Electrospun gelatin mats disclosed herein may optimally support the formation of astrocytes and endothelial cells for the production of a bilayer BBB model in a transwell format. Described is the formation of gelatin “biopaper” membranes, attachment to cell culture inserts, and culture conditions for a transwell BBB model based on this substrate. The trans-endothelial electrical resistance (TEER), molecular permeability, microscopic morphology, and immunohistochemistry (IHC) between biopaper cultures and PET based membranes are compared.

Each embodiment includes a biocompatible membrane. The membrane is generally not harmful to living tissue, including specifically human cells, human lung cells, human brain cells, human astrocytes, human brain microvascular endothelial cells, human small human small airway epithelial cells, human lung microvascular endothelial cells, or other cells to be used with the membrane. The membrane may be of any dimensions useful for the formation of tissues. For example, the membrane may be up to 1 mm thick. Suitable membrane materials include gelatin, collagen, polycaprolactone (PCL), polylactic acid (PLA), polyglycolic acid (PGA), and poly(DL-lactide-co-glycolide) (PLGA). The membrane material may be crosslinked, such as crosslinked gelatin. Generally, the membrane may have any dimensions or composition as described in U.S. Pat. No. 8,669,086.

The membrane may include a structural component, which is a solid material that defines the overall shape of the membrane. For example, a mat of woven or entangled fibers may be a structural component having the other biocompatible material permeated therein. Each of the components of the membrane may be either biodegradable (e.g. able to be broken down by biological means) or non-biodegradable. For example, the membrane may include a non-biodegradable structural component, such as fibers or polymer, and biodegradable material or polymer. In another embodiment, the structural component is also biodegradable, but at a slower rate than the other biodegradable material. In yet another embodiment, the membrane is free of any non-biodegradable structural component.

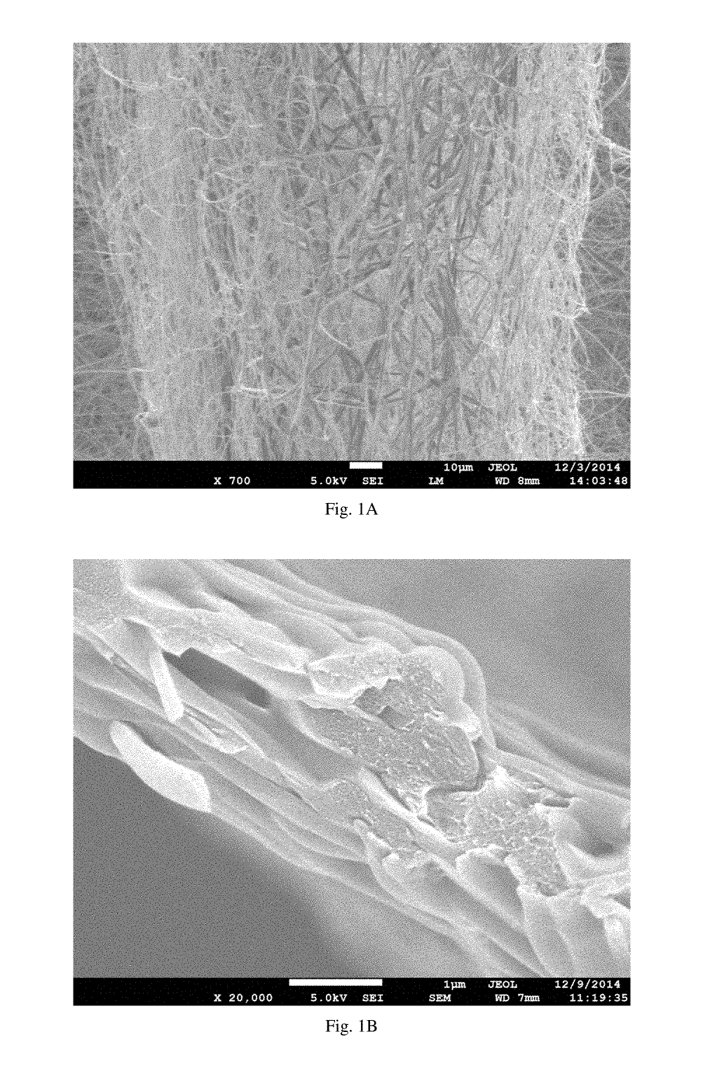

The membrane may be made by an electrospinning process. In electrospinning, fibers are spun from a liquid droplet by applying a high voltage to the droplet. An example electrospinning process is described below. The process may produce average nanofiber diameters of, for example, 100-400 nm. In another embodiment the polymeric nanofibers may have a variety of sizes, organized in layers, so that for example 100-400 nm fibers are on the outside of a triple layer mat where 800-1600 nm fibers are sandwiched between, providing mechanical characteristics and surface characteristics not achievable with a single layer or single range of fiber diameters. FIGS. 1A-B show cross-sections of such a triple layer biopaper before (FIG. 1A) and after (FIG. 1B) crosslinking. The different diameters may be produced by varying the electrospinning solution recipe or electrospinning parameters (voltage, flow rate, etc.).

In some embodiments, the membrane is attached to a rigid component having at least one hole. The membrane is attached so that it spans across the hole. The rigid component can act as a frame to hold the membrane substantially flat and to allow for mechanical stability during alignment and handling. Suitable materials for the frame include a cyclic olefin copolymer (COC), other polymer, metallic, or ceramic material. COC may be used because its glass transition temperature can be selected to allow for hot embossing of micro channels for perfusion of the supported tissue layer, but resist melting when autoclave sterilized.

The frame may have a single relatively large hole, or it may have a plurality of relatively smaller holes, with the membrane spanning each of the holes. The part of the frame between the holes may be the same material as the rest of the frame, or it may be a different material, which may be biodegradable.

The membrane may be attached to the frame by any means, including electrospinning the polymer directly onto the frame. Alternatively, a previously formed membrane may be heat sealed to the frame. It may be preferable for any crosslinking to occur after heat sealing in order to promote adhesion to the frame. The degree of crosslinking may be selected to provide for a desired biodegradation rate.

In some embodiments, living cells are placed onto or both sides of the membrane, even in the absence of a frame. The cells on each side may be of different types, and may include eukaryotic and prokaryotic cells. For example, one side may have human astrocytes and the other side human brain microvascular endothelial cells. In another example, one side has human small airway epithelial cells and the other side human lung microvascular endothelial cells. The cells may be placed on the membrane by any methods, such as the printing methods disclosed in U.S. Pat. No. 8,669,086.

In another embodiment, a non-degradable polymer can be spun on-top of the degradable polymer, or between two layers of degradable polymer fibers (as step 2 in a 3 step process). One possibility is to use the aliphatic polyamide 4.6 (PA 4.6) dissolved in a mixture of formic acid and acetic acid. A typical formulation would be employ 18% w/v PA 4.6 (MW: 80,000) dissolved in 50% formic acid and 50% acetic acid and gently stirred overnight (De Schoenmaker et al., “Electrospun Polyamide 4.6 Nanofibrous Nonwovens: Parameter Study and Characterization” Journal of Nanomaterials 2012, e860654). The solution is then loaded into a 20 mL syringe with a 1 mm ID needle. The distance from needle tip to collector plate is 12 mm and the electric potential between tip and collector is 25 kV. Flow rate is set at 4.5 mL/h. The time of deposition correlates to layer thickness may be as minimal as 30 seconds depending on how much non-degradable support is required.

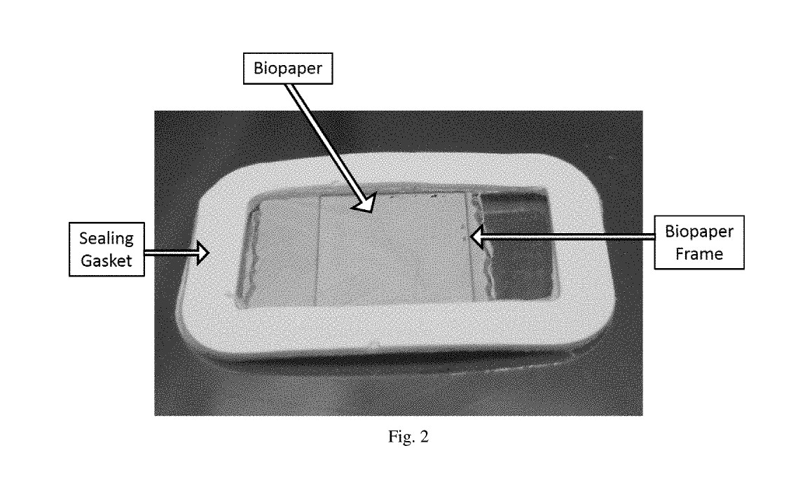

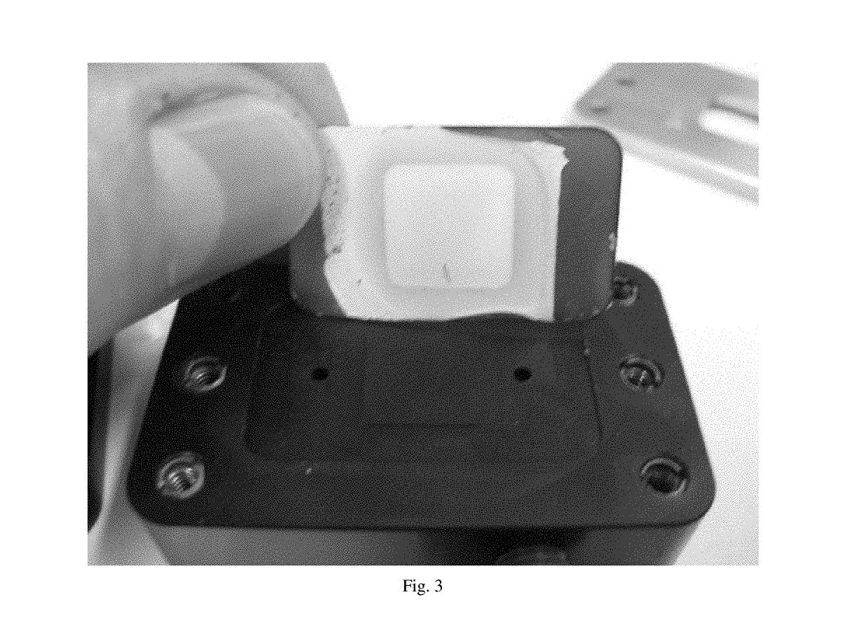

One or more of the membranes and frames may be used in the bioreactor disclosed in U.S. patent application Ser. No. 15/367,890. FIG. 2 shows a gelatin biopaper attached to a COC frame with expanded PTFE gasket. This may be a cell culture consumable that would be used with a reusable companion bioreactor. FIG. 3 shows a biopaper attached to a stainless steel frame, shown with a bioreactor into which it is placed.

Electrospun gelatin mats may have several advantages as cell culture substrates over commercially available cell culture insert materials such as the ability for cells to remodel and degrade the material over time. Additionally, the mechanical properties of the electrospun gelatin biopapers are closer to in vivo than materials used in commercial cell culture inserts. In vivo brain tissue has an estimated Young's modulus around 8-10 kPa (Soza et al., “Determination of the elasticity parameters of brain tissue with combined simulation and registration” Int. J. Med. Robot., vol. 1, no. 3, pp. 87-95, September 2005), while PET has a Young's modulus of 2 GPa. While still several orders of magnitude higher, the electrospun gelatin biopapers with a Young's modulus around 3.4 MPa are significantly closer to in vivo rigidity compared to PET. Similarly, the thickness of the PET and biopaper membranes are 10 μm and 4.5 μm, respectively, while in vivo basement membrane thickness is less than 1 μm (Carlson et al., “Ultrastructural morphometry of capillary basement membrane thickness in normal and transgenic diabetic mice” Anat. Rec. A. Discov. Mol. Cell. Evol. Biol., vol. 271A, no. 2, pp. 332-341, April 2003; Hawkins et al., “Structure of the blood-brain barrier and its role in the transport of amino acids,” J. Nutr., vol. 136, no. 1, p. 218S-226S, 2006).

Other potential advantages of the biopaper include: 1) ability to tune the degradation derived from variation of genipin crosslinker content in spinning solution and time mats are soaked in genipin solution, 2) more permeability to soluble molecules than conventional etched polymer membranes, 3) more porosity allowing increased cell-cell contact than conventional etched polymer membranes, 4) minimal thickness of ˜5 μm is much thinner than comparable membranes, made possible by strength and elasticity, 5) mechanical properties and degradation may be tuned by varying fiber size, thickness and/or cross-linking time and methods.

The following examples are given to illustrate specific applications. These specific examples are not intended to limit the scope of the disclosure in this application.

Using a modified protocol based on Panzavolta et al., “Electrospun gelatin nanofibers: Optimization of genipin cross-linking to preserve fiber morphology after exposure to water” Acta Biomater., vol. 7, no. 4, pp. 1702-1709, 10 mL of a gelatin-genipin solution was prepared by dissolving type B gelatin from bovine skin (Sigma Aldrich, Saint Louis, Mo., USA) in 70% (vol %) acetic acid (Sigma Aldrich) in distilled water to final concentrations of 30% or 15% (w/v), which was then stirred for 60 min. Genipin (Wako Chemicals, Richmond, Va., USA) equal to 3% of the gelatin weight used was then dissolved in 0.5 mL of ethanol and 1 mL of 1× phosphate-buffered saline (PBS) and added to the solution in order to stabilize the electrospun gelatin fiber morphology after exposure to aqueous solutions such as cell culture media. The amount of gelatin initially dissolved in the solution yielded 15% (w/v) and 30% (w/v) final concentrations after the addition of the 1.5 mL genipin solution. The solution was then transferred to a 20 mL plastic syringe (Thermo, Rockwood, Tenn., USA) with a 22-gauge stainless steel blunt-ended needle (Jensen Global, Santa Barbara, Calif., USA). The spinning solution was stored in this needle until electrospinning. Storage times between 30 minutes and 72 hours from solution mixing to electrospinning were tested and characterized.

Electrospun biopapers were created and collected using a custom electrospinning setup consisting of a Matsusada Precision AC100-240V high-voltage power supply (Kusatsu-City, Shiga, Japan), and a New Era Pump NE-300 syringe pump (Farmingdale, N.Y., USA) with the syringe and stainless steel blunt-ended needle. The power supply electrode was connected to the needle and positioned 15 cm from a grounded circular stainless steel plate (10 cm diameter) covered with non-stick aluminum foil. The syringe pump was set to a flow rate of 5 μL/min and a voltage of 15 kV was applied to the electrode for a total of 30 min to produce each electrospun mat. Electrospun mats were placed in a Sanplatec Dry Keeper desiccator (Kita-Ku, Osaka, Japan) for at least 24 hours.

Attachment to cell culture inserts—Transwell inserts are a standard cell culture device used for culturing bilayer tissues models where two different cell types are grown on opposite sides of a porous membrane. The electrospun gelatin biopapers described here can be used in such a configuration.

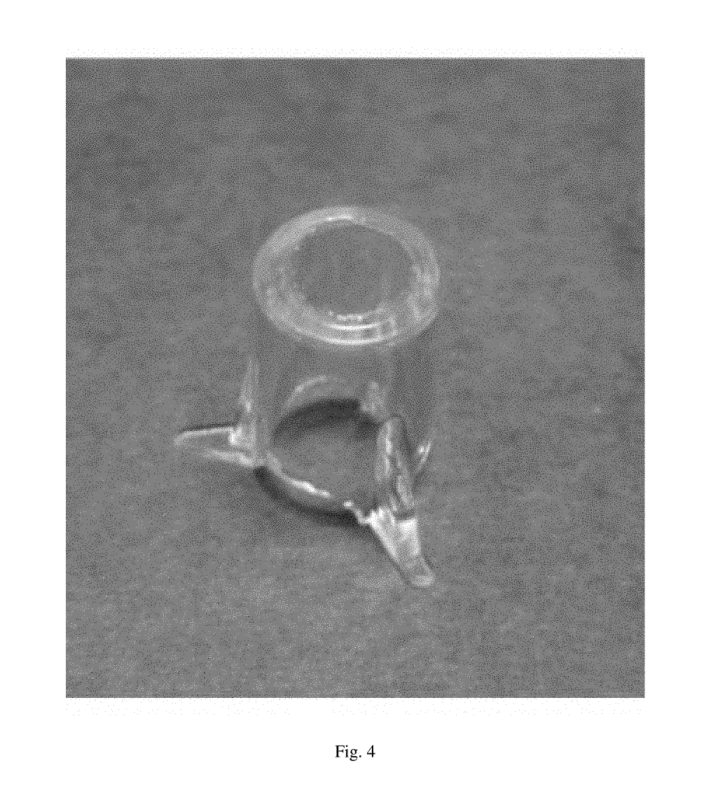

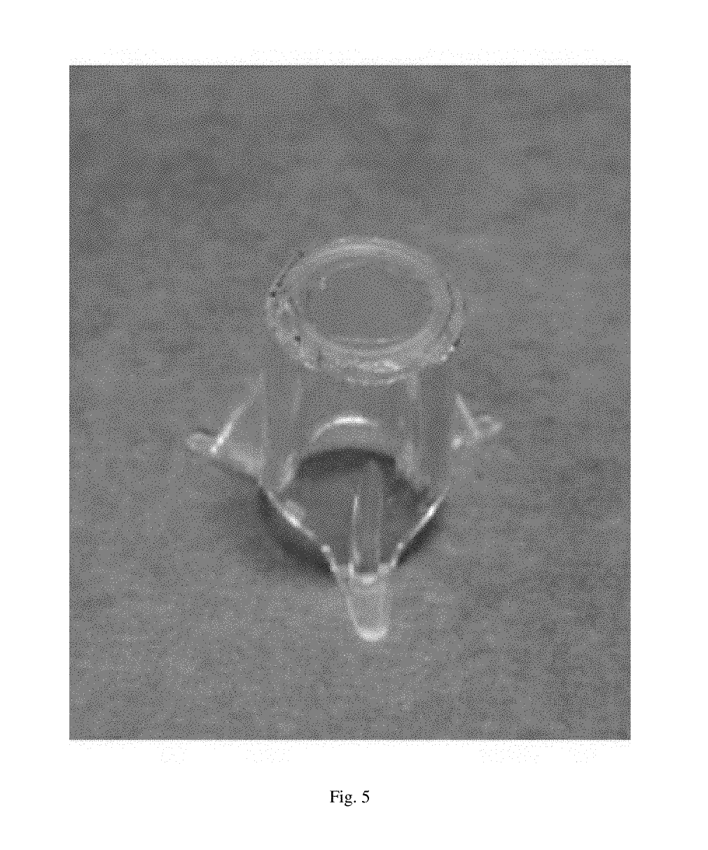

To prepare the electrospun gelatin biopapers for cell culture they were attached to hanging cell culture inserts. Using a soldering iron, the polyethylene terephthalate (PET) membranes of Millicell Cell Culture Inserts (24 well, 1 μm pore size, Millipore, Billerica, Mass., USA) were melted away until a thin ring roughly 1 mm in diameter remained around the circumference. Biopapers were then cut to size and placed over the opening (FIG. 4). The edges were then heat-sealed by tracing a soldering iron around the circumference of the insert using the soldering iron, and removing excess biopaper (FIG. 5).

Attachment to cell COC frames—The biopapers describe in U.S. Pat. No. 8,669,086 included a frame which provided mechanical support to the structure to make handling, printing, stacking, and alignment of multiple layers possible. The design disclosed herein expands upon that implementation. Frames were cut via laser mill from Cyclic Olefin Co-polymer (COC) sheets 0.002″ to 0.01″ thick. The gelatin fiber mats can then be attached to the COC frame by heat, ultrasonic welding, or chemical attachment (glue, crosslinking). Alternatively the fibers can be spun directly onto the COC frames and the bond strengthened by previously mentioned methods. The framed biopaper can also be fitted with the gaskets which allow it to seal into a bioreactor to be directly attached to the biopaper frame, making a convenient one time use consumable cell culture substrate which is used in conjunction with a compatible bioreactor. The biopaper is composed of electrospun gelatin, which can be optimized for a variety of cell culture applications by tuning fiber diameter, biopaper thickness, strength, and degradation rate. This customization is achieved by controlling the solution composition, electrospinning parameters (voltage, distance, pump rate), and crosslinking methods.

Cross-linking methods—Two cross-linking methods were compared: glyceraldehyde vapor based on Sisson et al., “Fiber diameters control osteoblastic cell migration and differentiation in electrospun gelatin” J. Biomed. Mater. Res. A, vol. 94, no. 4, pp. 1312-1320, September 2010, and soaking in a genipin/ethanol solution based on Panzavolta et al., “Electrospun gelatin nanofibers: Optimization of genipin cross-linking to preserve fiber morphology after exposure to water” Acta Biomater., vol. 7, no. 4, pp. 1702-1709. For glyceraldehyde vapor cross-linking, D,L-glyceraldehyde (Sigma Aldrich) was dissolved in 70% ethanol at a final concentration of 1% (w/v). This solution was placed in a petri dish in the bottom of a sealed container. Biopapers were placed in the container, but not in contact with the solution, for a total of 24 hours. Biopapers were then rinsed with water for 4 hours.

For genipin solution cross-linking, a 5% (w/v) genipin solution was prepared by dissolving genipin in ethanol. The biopaper cell culture inserts were placed in 24 well plates and soaked with the genipin solution (about 500 mL per well). The plates were sealed with parafilm and allowed to cross-link for 4 or 7 days at 37° C. The biopapers were then rinsed with water and dried prior to imaging. Upon drying, it was observed that the biopapers became brittle and susceptible to breakage so they were stored in water at 4° C. until needed.

Characterization—Scanning electron microscopy (SEM) was performed to evaluate fiber diameter and porosity of electrospun mats before and after cross-linking. Samples were sputter-coated with 5 nm of gold using an AJA Sputter Deposition System (North Scituate, Mass., USA) and observed using a JEOL JSM-7600F Field Emission SEM (Peabody, Mass., USA) or a Carl Zeiss SMT Supra55 scanning electron microscope (Maple Grove, Minn., USA) each operated at 5 kV. All samples were mounted using carbon tape on aluminum SEM studs. An average fiber diameter from 10 samples was calculated using ImageJ analysis software.

A TA.XT Texture Analyzer (Texture Technologies, Hamilton, Mass., USA) was used to determine the Young's Modulus of the biopapers. An STIL CHR450 high resolution sensor with an XS-300 Xenon source was used to estimate the thickness of the biopapers.

Cell culture—Primary human astrocytes (HAs) were obtained from ScienCell Research Laboratories (Carlsbad, Calif., USA) and maintained according to manufacturer directions. Human brain microvascular endothelial cells (HBMECs) were obtained from Cell Systems (Kirkland, Wash., USA). Human small airway epithelial cells (SAEC) and Human Lung Microvascular Endothelial Cells (HMVEC-L) were obtained from Lonza (Walkersville, Md., USA) and maintained according to manufacturer directions.

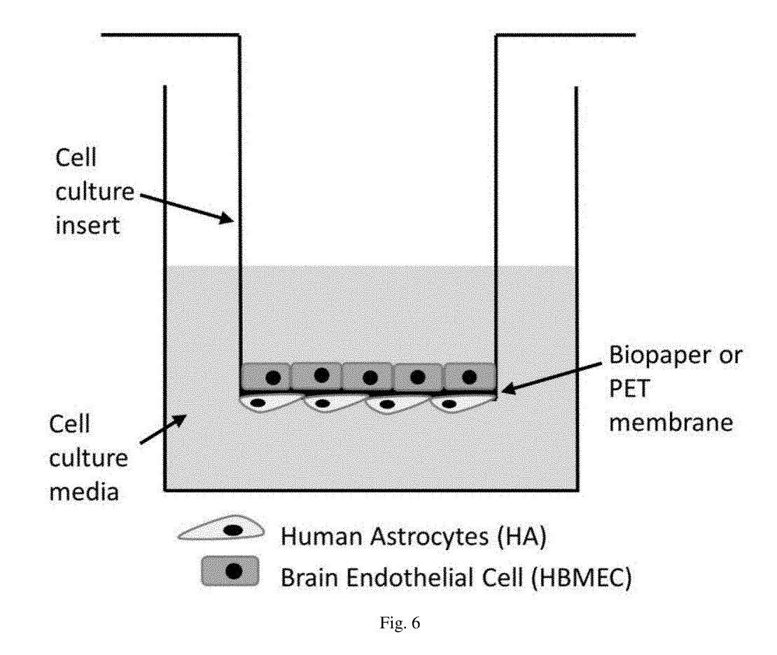

To generate the in vitro bilayer human blood-brain barrier (BBB) model, cell culture inserts with either the standard PET membrane or the attached biopaper membranes were first flipped upside-down (on the cover of a 24-well plate). 50 μL of HA medium containing 25,000 HAs were added to the bottom side of each insert. The well plate was then used to cover the inserts which were placed in an incubator at 37° C. for 2 hours to allow for cell attachment. After 2 hours the plates were righted and 1 mL of HA medium was added to each well. The following day, 500 μL of HBMEC medium containing 25,000 HBMECs was added to the top of the cell culture insert. Only the first passage of cells was used to minimize phenotypic changes from experiment to experiment. The medium was changed every 2-3 days and the cultures were maintained up to 21 days. A schematic of the BBB model is shown in FIG. 6.

Cell staining and image acquisition—After 10 days BBB model cultures were fixed in 4% (w/v) paraformaldehyde and stained for the endothelial cell marker platelet endothelial cell adhesion molecule (PECFiAM-1 or CD31) and the astrocyte marker glial fibrillary acidic protein (GFAP). Following fixation, cell membranes were permeabilized with 0.25% Triton-X-100 and blocked with 3% bovine serum albumin for 4 hours. Cultures were incubated with mouse anti-CD31 antibodies (1:50, Invitrogen, Grand Island, N.Y., USA) and rabbit anti-GFAP (1:200, Invitrogen) for 4 hours at room temperature and rinsed several times with 1×PBS. BBB cultures were then incubated with AlexaFluor568 conjugated anti-mouse and AlexaFluor488 conjugated anti-rabbit antibodies (1:200, Sigma) for 2 hours at room temperature. After several rinses in 1×PBS, membranes were removed from the cell culture inserts and mounted to glass slides with cover glass for imaging. Samples were imaged using an AIR confocal microscope (Nikon Instruments, Tokyo, Japan). Images were acquired and volume-rendered using NIS Elements software (Nikon).

Characterization of cell morphology and barrier integrity—Electrospun gelatin biopaper membranes were developed as a cell culture substrate for in vitro bilayer models of human blood-brain barrier tissue. Fiber diameter and cross-linking methods were optimized to maintain fiber morphology after exposure to liquid and to promote co-culture of primary human astrocytes and primary human brain microvascular endothelial cells. Morphology and barrier properties of cell cultures on biopaper membranes and standard cell culture insert membranes (PET) were characterized.

Barrier integrity was assessed by measuring the trans-endothelial electrical resistance using an EVOM2 epithelial voltohmmeter for TEER with an Endohm 6 mm chamber (World Precision Instruments, Sarasota, Fla., USA). To calculate the Ω-cm², the resistance measurement of a blank insert was subtracted from the resistance measurement of the culture model and multiplied by the cell culture area. Average TEER values were monitored for 21 days across three independent experiments (total n=12). A student's T-test was performed at each timepoint to determine statistical significance between TEER values of cultures on the different materials. P-values less than 0.05 were considered to be significant.

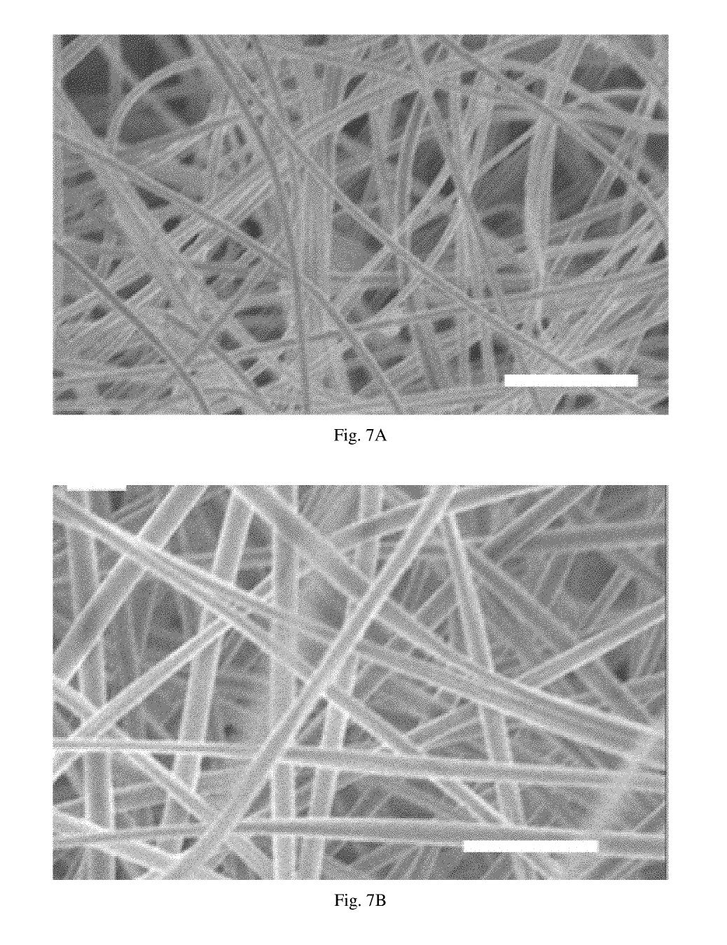

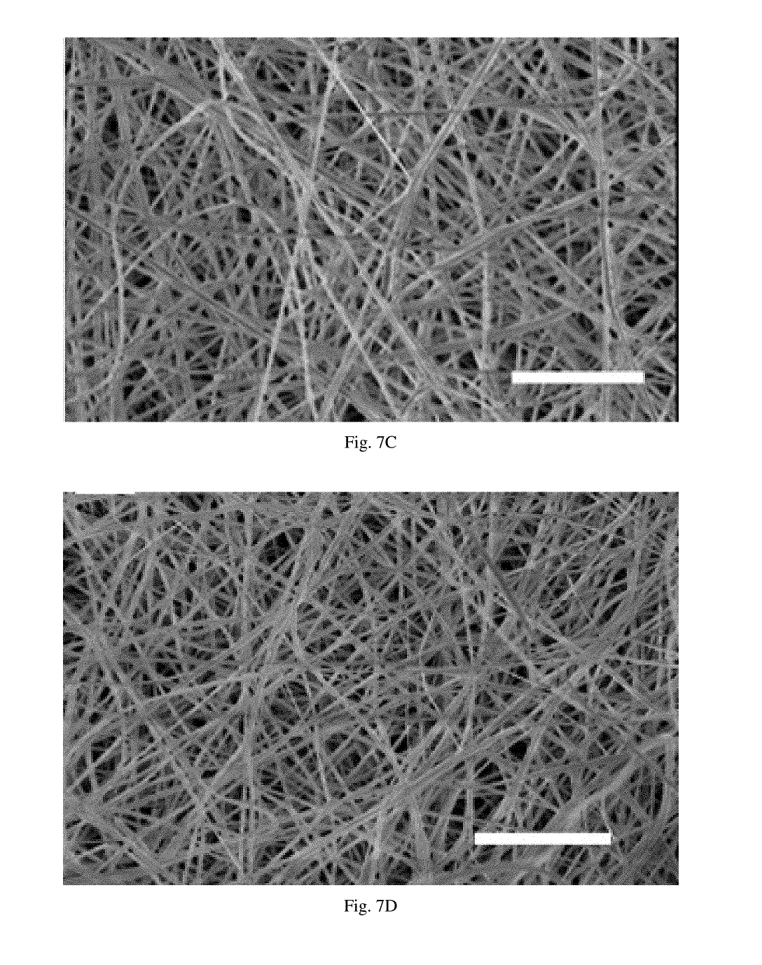

Effect of solution preparation and cross-linking methods on fiber diameter and morphology—Biopapers consisting of randomly aligned fibers were obtained by electro spinning solutions of gelatin and genipin in 70% (w/v) acetic acid in water for 30 minutes. Genipin, a cross-linking agent for electrospun gelatin, was included in the initial solution at a concentration of 3% of the gelatin concentration (w/w) in order to stabilize the electrospun gelatin fiber morphology after exposure to aqueous solutions such as cell culture media (Panzavolta). FIGS. 7A-D show electrospun gelatin fiber diameters from solutions of 30% (FIG. 7A) and 15% (FIG. 7C) gelatin with 3% genipin, which had average fiber diameters of 487±109 nm and 112±22 nm respectively, when electrospun 30 min after the addition of genipin to the solution. It was observed that increasing the time between genipin addition and electrospinning resulted in thicker fibers, likely due to cross-linking of the gelatin in the solution increasing the overall solution viscosity. When gelatin solutions were electrospun 24 hours after the addition of genipin, fiber diameters for 30% (FIG. 7B) and 15% (FIG. 7D) gelatin increased to 567±92 nm and 133±15 nm respectively. Electrospinning 48 or 72 hours after the addition of genipin to the solution resulted in no further increase in fiber diameter. To facilitate reproducibility when electrospinning multiple batches of biopapers in one day, biopapers were electrospun 24 hours after the addition of genipin for remaining experiments.

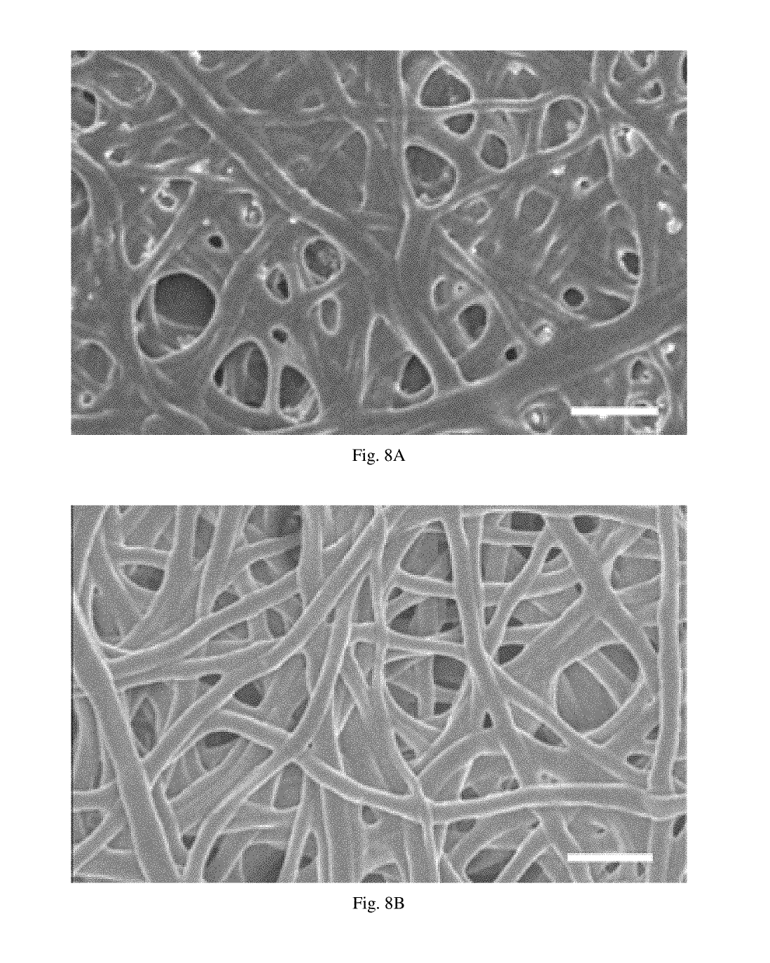

Prior to additional cross-linking, biopapers were cut to size, removed from the collectors, and attached to commercial cell culture inserts with the original membranes removed. Briefly, the biopaper was positioned overhanging the opening in the bottom of the insert and heat-sealed by tracing a soldering iron around the edge of the insert and biopaper. Further cross-linking via two different methods was compared. FIGS. 8A-B show 30% electrospun gelatin biopapers cross-linked either by exposure to vapor from a 0.5% glyceraldehyde in 70% ethanol solution (w/v) for 24 hours (FIG. 8A), or by soaking in a 5% genipin in ethanol solution for 7 days (FIG. 8B). After cross-linking, biopapers were rinsed with water and allowed to soak for at least 4 hours before imaging. In both cases some fusion of fibers occurred resulting in increased fiber diameters (1667±345 nm and 936±154 nm for glyceraldehyde and genipin cross-linking, respectively). However, fiber morphology was better maintained when the biopapers were cross-linked with the genipin solution.

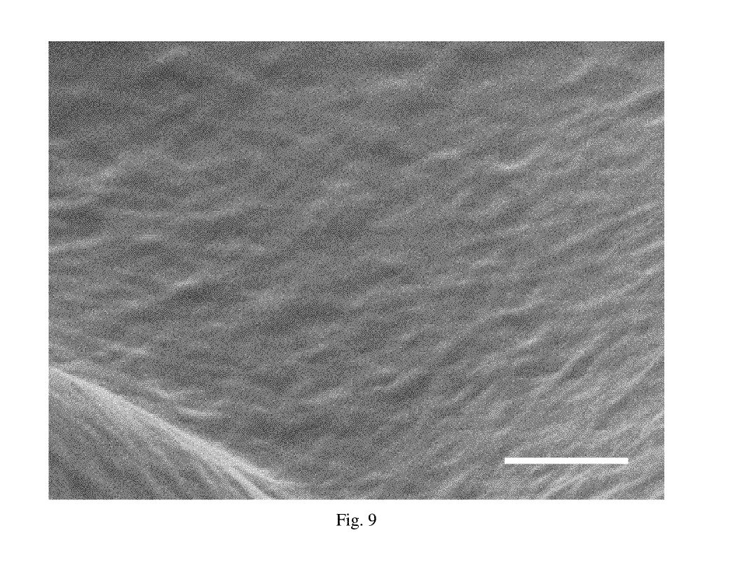

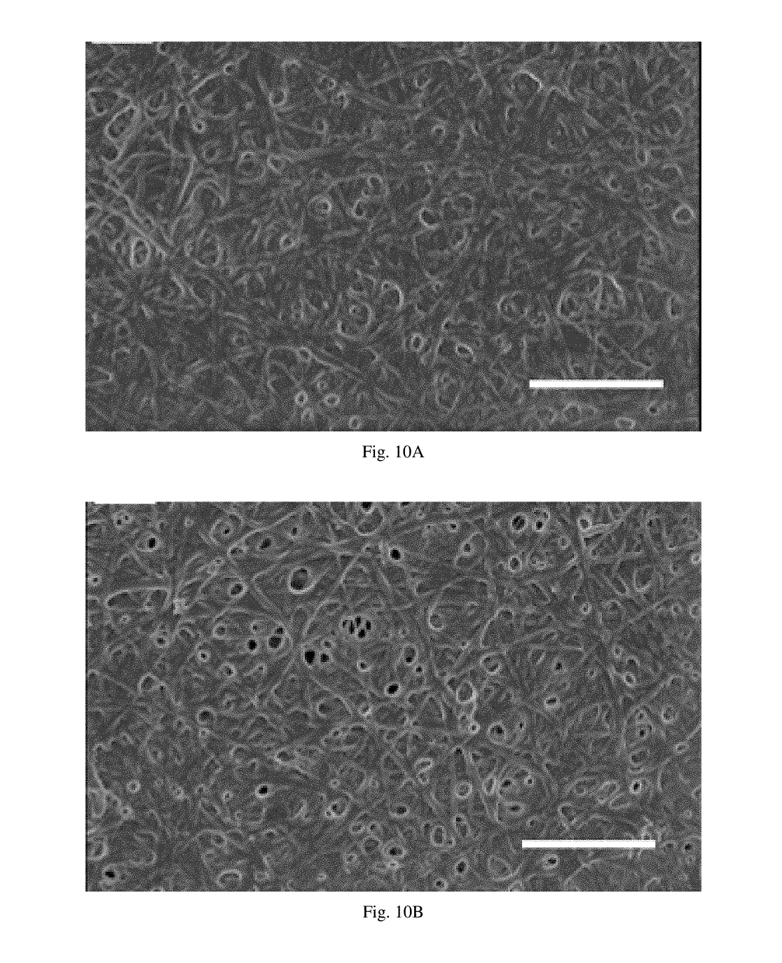

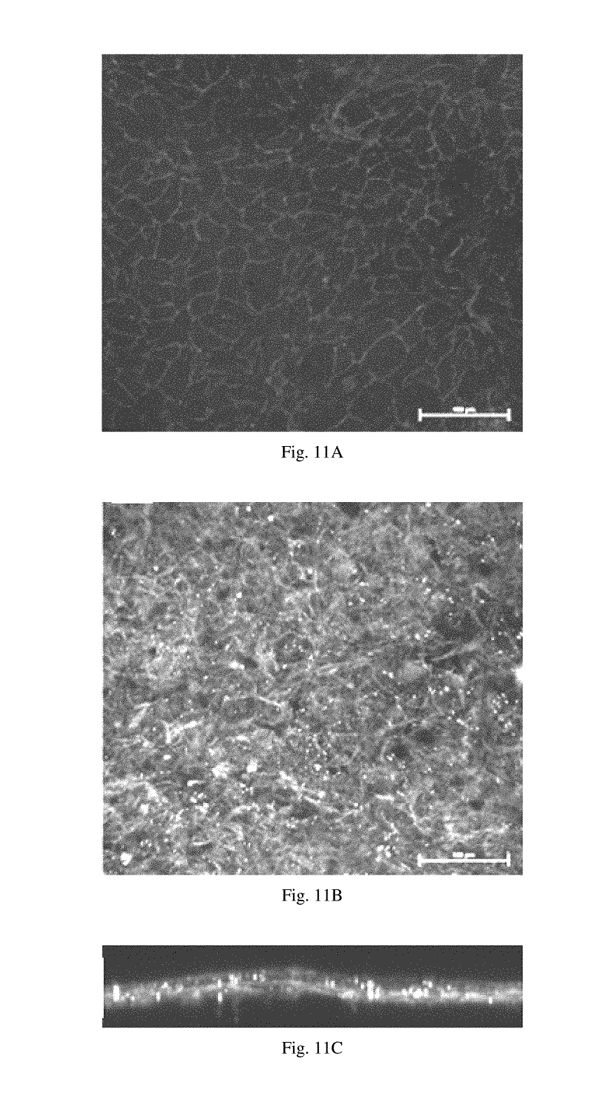

In order to verify that the inclusion of genipin in the initial electrospinning solution was necessary, a 30% gelatin solution without genipin was electrospun and cross-linked by soaking in a 5% genipin solution for 7 days. FIG. 9 shows that after exposure to water, fiber morphology was not maintained. FIGS. 10A-B show the fiber morphology of biopapers soaked in genipin for 4 days (FIG. 10A) and 7 days (FIG. 10B) and exposed to water. Of the two soaking durations, fiber morphology was better maintained after soaking for 7 days.

Optimization efforts determined that the electrospun 15% gelatin biopapers cross-linked by soaking in a 5% genipin solution for 7 days was optimal. The fiber diameter was 196±46 nm and the biopaper was found to have an overall thickness of 4.5±1.5 μm when dry. The Young's Modulus was determined to be 3.4±1.2 MPa using a texture analyzer.

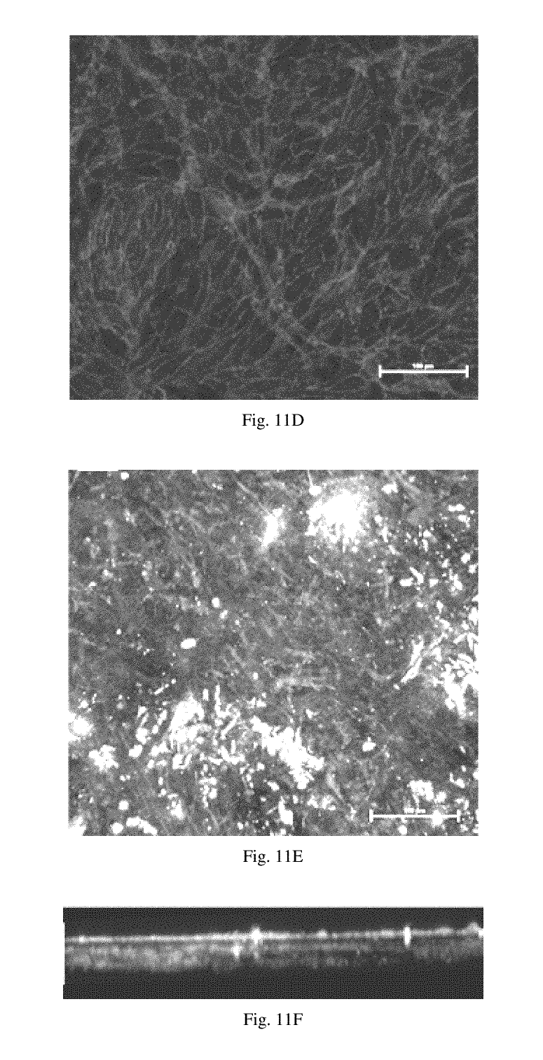

Comparison of in vitro bilayer human BBB models on biopaper and standard cell culture inserts—An in vitro bilayer co-culture to model human BBB tissue was generated by co-culturing primary human astrocytes on the bottom of the biopaper membranes with primary human brain microvascular endothelial cells on top of the membranes. Direct comparisons of these models were made to cultures on standard PET cell culture inserts. Cultures were maintained for 21 days. As shown in FIGS. 11A-F, confocal microscopy was used to assess the morphology of cells grown on 15% electrospun gelatin biopapers and PET membranes. Astrocytes were positive for the astrocyte marker GFAP and the endothelial cells expressed the endothelial cell junction marker PECAM-1 along the cellular junctions. While cell morphology appeared similar in both cases, volume-rendered side views (FIG. 5C, F) demonstrate that the gap between the two cell types was much more pronounced for the PET membrane compared to the biopaper membranes.

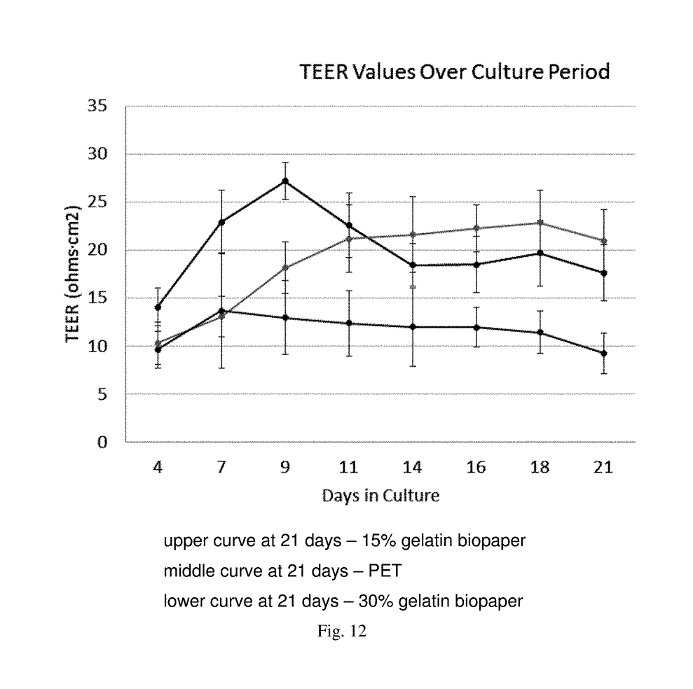

TEER values were measured to assess barrier integrity. As shown in FIG. 12, TEER values for BBB models on PET peaked after 9 days in co-culture around 27 Ω-cm² and then leveled off around 18 Ω-cm². TEER values for BBB models on the 15% electrospun gelatin biopapers rose more slowly, peaking around 22 Ω-cm² but maintaining this peak value throughout the 21 day culture period. BBB models were also grown on 30% electrospun gelatin biopapers but TEER values were consistently lower than models on PET or 15% electrospun gelatin biopapers. The difference in TEER values was statistically significant (p<0.05) between PET and 15% or 30% gelatin biopapers at every timepoint after 7 days. Based on these results, it was determined that the electrospun 15% gelatin biopapers cross-linked by soaking in a 5% genipin solution for 7 days was optimal. The fiber diameter was 196±46 nm and the biopaper was found to have an overall thickness of 4.5±1.5 μm when dry. The Young's Modulus was determined to be 3.4±1.2 MPa using a texture analyzer.

Permeability of the in vitro bilayer human BBB models to fluorescein isothiocyanate (FITC) labeled dextrans of different molecular weights (10, 20, 40, and 70 kD) was investigated to assess barrier integrity at different time points (4, 12, and 21 days) using a protocol based on Artusson “Epithelial transport of drugs in cell culture. I: A model for studying the passive diffusion of drugs over intestinal absorbtive (Caco-2) cells” J. Pharm. Sci. 1990 Jun. 1; 79(6):476-82. 50 μL samples of medium were collected from the bottom chamber at 1, 5, 10, 20, 30, and 60 min after the addition of the dextran and placed into a well of a 96 well plate. Each time a sample was taken an equal volume of fresh medium was replaced in the bottom chamber. The fluorescent intensities were measured using a BioTek Synergy HT microplate reader (Winooski, Vt., USA) with BioTek Gen5 software. Graphs generated in Microsoft Excel were used to calculate the change in intensity over time. A standard curve was used to verify that fluorescent intensity scaled linearly with concentration and the sample intensity was then converted to concentration. The permeability coefficient was calculated according to Equation (1), where P (cm/s) is the apparent permeability coefficient, dQ/dt (mg/s) is the rate of FITC-dextran concentration change over time, A (cm²) is the culture surface area, and Co (mg/mL) is the initial concentration of FITC-dextran in the apical chamber.

P=(dQ/(dt)(1/AC₀)) (1)

The averages and standard deviations from the five samples of BBB on PET and biopaper for each of the dextran molecular weights and at each time point were calculated as well as the permeability of PET and biopaper membranes without cells. As shown in Table 1, bilayers grown on the 15% electrospun gelatin biopapers started out roughly 100 times more permeable than bilayers grown on PET membranes. By day 12 the permeability between the two membranes was similar and these levels were maintained through the 21 day culture period. As expected, permeability to dextrans with lower molecular weights was greater than permeability to dextrans with higher molecular weights.

TABLE 1
permeability of in vitro human BBB co-cultures on biopaper
and PET to FITC-labeled dextran at different time points

| | | | |
|---|---|---|---|
| Blank | Day 4 | Day 12 | Day 21 |

Permeability to 10 kD FITC-Dextran (×10⁻⁶ cm/s)

| | | | | |
|---|---|---|---|---|
| PET | 321 ± 168 | 4.70 ± 2.87 | 2.07 ± 0.30 | 2.47 ± 0.62 |
| Biopaper | 556 ± 228 | 808 ± 156 | 7.27 ± 4.07 | 1.39 ± 0.97 |

Permeability to 20 kD FITC-Dextran (×10⁻⁶ cm/s)

| | | | | |
|---|---|---|---|---|
| PET | 160 ± 108 | 2.63 ± 1.82 | 1.62 ± 1.11 | 1.01 ± 0.53 |
| Biopaper | 485 ± 166 | 227 ± 53 | 1.82 ± 0.93 | 0.56 ± 0.23 |

Permeability to 40 kD FITC-Dextran (×10⁻⁶ cm/s)

| | | | | |
|---|---|---|---|---|
| PET | 116 ± 97 | 1.88 ± 0.92 | 1.03 ± 1.19 | 0.86 ± 0.45 |
| Biopaper | 364 ± 274 | 217 ± 49 | 1.43 ± 0.83 | 0.49 ± 0.19 |

Permeability to 70 kD FITC-Dextran (×10⁻⁶ cm/s)

| | | | | |
|---|---|---|---|---|
| PET | 103 ± 17 | 1.15 ± 0.40 | 0.91 ± 0.61 | 0.51 ± 0.53 |
| Biopaper | 187 ± 248 | 189 ± 57 | 1.03 ± 0.17 | 0.39 ± 0.20 |

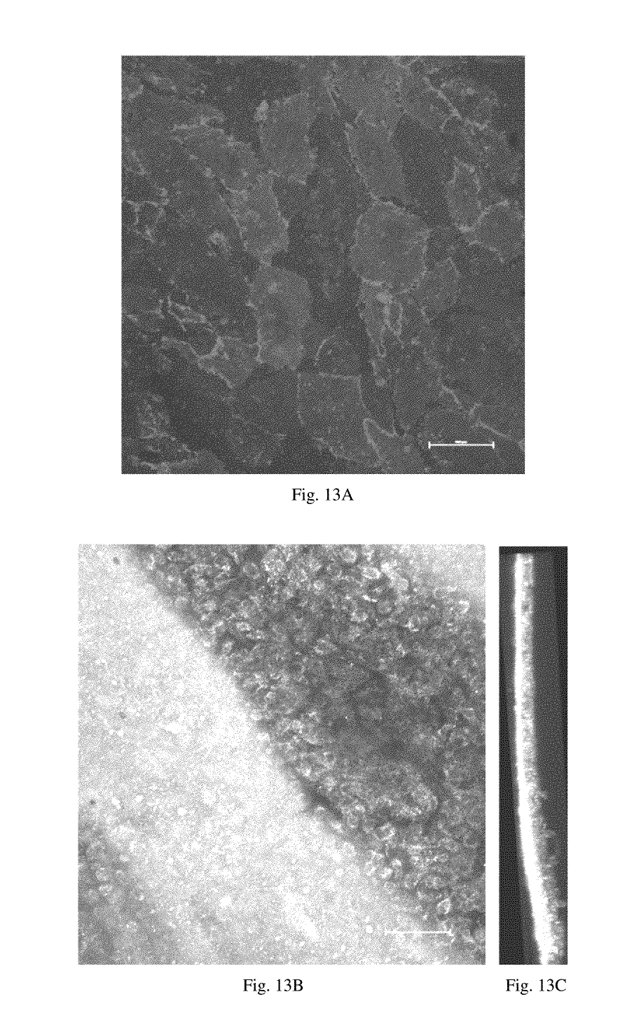

FIGS. 13A-C show another example using human lung microvascular endothelial cells (HMVEC-L) (FIG. 13A) on one side and human small airway epithelial cells (SAEC) (FIG. 13B) on the other side.

Discussion—To characterize the in vitro bilayer human BBB models, cells grown on electrospun gelation biopapers were compared to cells grown on standard PET cell culture inserts. In both cases, cell morphology was similar with the HBMECs staining for the endothelial cell marker PECAM-1 around the cell junctions, and the HAs staining for the astrocyte marker GFAP. However, the endothelial cells appear slightly more spread on the PET membranes. This may indicate that the gelatin biopaper supports improved cell growth or that cells spread more on PET, perhaps due to greater stiffness (Engler et al., “Substrate Compliance versus Ligand Density in Cell on Gel Responses” Biophys. J. 2004 January; 86(1):617-28). Further studies were performed to compare the barrier integrity of cultures on biopaper and PET. Additionally, the gap between the endothelial cells and astrocytes was larger for the PET membrane compared to the biopaper membranes. This smaller gap more closely mimics a thin basement membrane and, coupled with increased porosity and the ability of gelatin to be remodeled by cells, provides for more cell-cell contact between the endothelial cells and astrocytes, a feature shown to be important in the integrity of the blood-brain barrier (Hurwitz et al., “Human fetal astrocytes induce the expression of blood-brain barrier specific proteins by autologous endothelial cells” Brain Res. 1993 Oct. 22; 625(2):238-43; Stoll et al., “Dynamic imaging of T cell-dendritic cell interactions in lymph nodes” Science 2002 Jun. 7; 296(5574):1873-6; Fromm et al., Molecular Structure and Function of the Tight Junction: From Basic Mechanisms to Clinical Manifestations. John Wiley & Sons; 2009. 357 p.)

Barrier integrity was examined by comparing TEER measurements of cultures on biopaper and PET. Initially, 30% electrospun gelatin biopapers were used and it was found that TEER values for cells grown on these membranes were between 10-15 Ω·cm², while TEER values for cells grown on PET had a maximal value around 27 Ω·cm² after 9 days and averaged between 18-20 Ω·cm² later in the culture period. When the gelatin concentration was reduced to 15%, TEER values were similar to the observed TEER values on PET, reaching and maintaining values between 20-23 Ω·cm² throughout the culture period. It has been demonstrated that substrate porosity influences TEER readings of cells cultured on permeable membranes (Lo et al., “Cell-substrate contact: another factor may influence transepithelial electrical resistance of cell layers cultured on permeable filters” Exp. Cell. Res. 1999 Aug. 1; 250(2):576-80). Additionally, several studies have shown that fiber diameter can influence cell behavior, including cell spreading, with smaller fiber diameter causing increased endothelial cell spreading (Whited et al., “The influence of electrospun scaffold topography on endothelial cell morphology, alignment, and adhesion in response to fluid flow” Biotechnol. Bioeng. 2014 Jan. 1; 111(1):184-95; Hodgkinson et al., “Electrospun silk fibroin fiber diameter influences in vitro dermal fibroblast behavior and promotes healing of ex vivo wound models” J. Tissue Eng. [Internet]. 2014 Sep. 18 [cited 2015 Jun. 5]; 5. Available from: http://www.ncbi.nlm.nih.gov/pmc/articles/PMC4221927/; Christopherson et al., “The influence of fiber diameter of electrospun substrates on neural stem cell differentiation and proliferation” Biomaterials 2009 February; 30(4):556-64; Gnavi et al., “The influence of electrospun fibre size on Schwann cell behaviour and axonal outgrowth” Mater. Sci. Eng. C 2015 Mar. 1; 48:620-31. Overall, the TEER values of these in vitro models, whether on PET or biopaper, were much less than in vivo estimates of at least 1000 Ω·cm², yet were within the reported range for in vitro BBB models (Li et al., “Permeability of endothelial and astrocyte cocultures: in vitro blood-brain barrier models for drug delivery studies” Ann. Biomed. Eng. 2010 August; 38(8):2499-511; Deli et al., “Permeability Studies on In Vitro Blood-Brain Barrier Models: Physiology, Pathology, and Pharmacology” Cell Mol. Neurobiol. 2005 Feb. 1; 25(1):59-127; Eigenmann et al., “Comparative study of four immortalized human brain capillary endothelial cell lines, hCMEC/D3, hBMEC, TY10, and BB19, and optimization of culture conditions, for an in vitro blood-brain barrier model for drug permeability studies” Fluids Barriers CNS 2013 Nov. 22; 10(1):33; Jong et al., “Traversal of Candida albicans across human blood-brain barrier in vitro” Infect. Immun. 2001 July; 69(7):4536-44; Gaillard et al., “Relationship between permeability status of the blood-brain barrier and in vitro permeability coefficient of a drug” Eur. J. Pharm. Sci. Off. J. Eur. Fed. Pharm. Sci. 2000 December; 12(2):95-102.

The permeability of the models was tested on PET and 15% electrospun gelatin biopaper to FITC-labeled dextran of different molecular weights (10, 20, 40, and 70 kDa). The apparent permeability of PET and biopaper inserts were measured to be on the order of 10⁻⁴ cm/s and were more permeable to smaller size dextran molecules than larger sizes. The blank biopaper membranes were more permeable than the PET membranes. 4 days after cells were added, the apparent permeability remained on the order of 10⁻⁴ cm/s for cells cultured on biopaper, but fell to 10⁻⁶ cm/s for cells on PET. However, by 12 days in culture the apparent permeability for cells on both biopaper and PET was on the order of 10⁻⁶ cm/s; this was maintained throughout the 21 day culture period. The permeability measurements followed a similar trend to the observed TEER values, with cells on PET reaching maximal TEER values earlier in the culture period than cells on biopaper. Despite being on the lower side of reported TEER values for in vitro BBB models in the literature, the permeability measurements are in good agreement with the 10⁻⁶ cm/s order of magnitude that is typical of in vitro BBB models (Li; Deli; Gaillard; Kuntz et al., “Stroke-induced brain parenchymal injury drives blood-brain barrier early leakage kinetics: a combined in vivo/in vitro study” J. Cereb. Blood Flow Metab. 2014 January; 34(1):95-107. In vivo measurements of BBB permeability are on the order of 10⁻⁷-10⁻¹⁰ cm/s.

Obviously, many modifications and variations are possible in light of the above teachings. It is therefore to be understood that the claimed subject matter may be practiced otherwise than as specifically described. Any reference to claim elements in the singular, e.g., using the articles “a”, “an”, “the”, or “said” is not construed as limiting the element to the singular.

What is claimed is:

1. An article comprising:
a biocompatible membrane made by electrospinning a polymer; and
a rigid component having a hole;
wherein the membrane is attached to the rigid component and spans the hole.

2. The article of claim 1, wherein the membrane comprises a biodegradable material.

3. The article of claim 2, wherein the membrane is free of a non-biodegradable structural component.

4. The article of claim 2, wherein the membrane comprises a structural component comprising a second biodegradable material that degrades slower that the biodegradable material.

5. The article of claim 1, wherein the membrane comprises non-biodegradable fibers.

6. The article of claim 1, wherein the membrane comprises a non-biodegradable structural component.

7. The article of claim 1, wherein the membrane comprises gelatin.

8. The article of claim 7, wherein the gelatin is crosslinked.

9. The article of claim 1;
wherein rigid components has a plurality of holes; and
wherein the membrane spans each of the plurality of holes.

10. The article of claim 9, wherein the portion of the rigid component between the plurality of holes is the same material as the portion of the rigid component surrounding the plurality of holes.

11. The article of claim 9, wherein the portion of the rigid component between the plurality of holes comprises a biodegradable material.

12. The article of claim 1, wherein the article further comprises:
living cells one or both surfaces of the membrane.

13. The article of claim 12, wherein the surfaces of the membrane comprise different types of cells.

14. The article of claim 13;
wherein human astrocytes are on a first surface of the membrane; and
wherein human brain microvascular endothelial cells are on a second surface of the membrane.

15. The article of claim 13;
wherein human small airway epithelial cells are on a first surface of the membrane; and
wherein human lung microvascular endothelial cells are on a second surface of the membrane.

16. The article of claim 1, wherein the rigid component is a frame comprising a cyclic olefin copolymer.

17. An article comprising:
a biocompatible membrane made by electrospinning a polymer; and
living cells on both surfaces of the membrane;
wherein the surfaces of the membrane comprise different types of cells.

18. The article of claim 17, wherein the membrane comprises a biodegradable material.

19. The article of claim 18, wherein the membrane further comprises non-biodegradable fibers.

20. The article of claim 17, wherein the membrane comprises gelatin.

21. The article of claim 20, wherein the gelatin is crosslinked.

22. The article of claim 17;
wherein human astrocytes are on a first surface of the membrane; and
wherein human brain microvascular endothelial cells are on a second surface of the membrane.

23. The article of claim 17;
wherein human small airway epithelial cells are on a first surface of the membrane; and
wherein human lung microvascular endothelial cells are on a second surface of the membrane.

24. An article comprising:
a biocompatible membrane comprising a polymer that is free of a non-biodegradable structural component; and
a rigid component having a hole;
wherein the membrane is attached to the rigid component and spans the hole.

25. A method comprising:
providing a biocompatible membrane made by electrospinning a polymer; and
attaching the membrane to a rigid component having a hole;
wherein the membrane spans the hole.

26. The method of claim 25, further comprising:
electrospinning the polymer to form the membrane.

27. The method of claim 25, wherein attaching the membrane comprises heat sealing the membrane to the rigid component.

28. The method of claim 25, further comprising:
crosslinking the electrospun polymer by a method that results in a predetermined degradation rate of the membrane.

29. The method of claim 25, further comprising:
depositing living cells one or both surfaces of the membrane.

30. The method of claim 29, wherein different types of cells are deposited on each surface of the membrane.

31. A method comprising:
providing a biocompatible membrane made by electrospinning a polymer; and
depositing living cells both surfaces of the membrane;
wherein different types of cells are deposited on each surface of the membrane.

32. The method of claim 31, further comprising:
electrospinning the polymer to form the membrane.

33. The method of claim 31, further comprising:
crosslinking the electrospun polymer by a method that results in a predetermined degradation rate of the membrane.
